(19) United States
(12) Patent Application Publication
(10) Pub. No.: US 2015/0374714 A1
(43) Pub. Date: Dec. 31, 2015

(54) GENERATION OF FUNCTIONAL DENDRITIC CELLS

(71) Applicant: UNIVERSITY OF GEORGIA RESEARCH FOUNDATION, INC., Athens, GA (US)

(72) Inventors: Shanta Dhar, Athens, GA (US); Sean Marrache, Portland, OR (US); Donald A. Harn, Athens, GA (US); Smanla Tundup, Downers Grove, IL (US)

(21) Appl. No.: 14/767,063

(22) PCT Filed: Feb. 11, 2014
(86) PCT No.: PCT/US2014/015744

§ 371 Date: Aug. 11, 2015

Related U.S. Application Data
(60) Provisional application No. 61/763,408, filed on Feb. 11, 2013.

(51) Int. Cl. A61K 31/555; C08G 63/08; C12N 5/0784; A61K 41/00; A61K 47/48

(52) U.S. Cl. CPC A61K 31/555; A61K 41/0057; A61K 47/48915; A61K 47/48084; A61K 47/48215; A61K 41/0071; C12N 5/0639; C08G 63/08; C12N 2501/999; C12N 2529/10

(57) ABSTRACT

Nanoparticles containing a photosensitizer configured to generate a reactive oxygen species when exposed to an appropriate wavelength of light can be used to enhance immunogenicity of cancer cells, such as breast cancer cells. Such enhanced immunogenicity cancer cells, or supernatants thereof, can be used to activate dendritic cells or cause dendritic cells to produce INF-gamma. Nanoparticles having mitochondria-targeting moieties are more effective at enhancing the immunogenicity of the cancer cells, or causing the dendritic cells to produce IFN-gamma, than nanoparticle lacking mitochondria-targeting moieties or free photo sensitizer.

### RELATED APPLICATION

This application claims the benefit of U.S. Provisional Patent Application No. 61/763,408, filed on Feb. 11, 2013.

### STATEMENT OF GOVERNMENTAL SUPPORT

This invention was made with government support under grant number P30GM092378, awarded by the National Institutes of Health of the United States government. The government has certain rights in the invention.

### FIELD

The present disclosure relates to nanoparticles configured to traffic agents to mitochondria and methods of use thereof, including diagnostic and therapeutic uses.

### BACKGROUND AND INTRODUCTION

Dysfunction of a host's immune system represents one of the major mechanisms by which tumors evade immunosurveillance. Tumors design strategies to successfully evade the host immune system, which strategies may target immune antitumor effector cells. Dysfunction and apoptosis of these immune antitumor effector cells in the tumor-bearing host creates an immune imbalance that cannot be corrected by immunotherapies aimed only at activation of anti-tumor immune responses. Reversal of existing immune dysfunction(s) and normalization of lymphocyte homeostasis in patients with cancer may be an important part of future cancer immunotherapy.

Despite aggressive management, survival for metastatic cancer patients remains low. As a result, systemic therapy has become an integral component of metastatic cancer management. However, the limited success of systemic chemotherapy underscores the need to develop new therapeutic strategies, and this urgency has resulted a number of alternatives like anti-tumor immunotherapy.

It is now well accepted that the immune system, when properly stimulated, can cause eradication of cancer cells. This field only experienced modest successes with nonspecific immune stimulants, such as interferon (IFN)-α and interleukin (IL)-2 for melanoma and renal cell carcinoma. Nonspecific immune stimulants also showed enhanced anti-tumor immunity with Bacille Calmette-Guerin for non-invasive bladder cancer. The success of active-specific immunotherapeutic approaches using protein/peptide, whole tumor cells, and dendritic cells (DCs) as vaccines has been sporadic and unpredictable for all tumor types. However, the active-specific stimulation of the host's own immune system holds great promise for achieving non-toxic and durable antitumor responses. Sipuleucel-T is the first immune-based therapeutic cancer vaccine to receive approval from the U S Food and Drug Administration (FDA), which is used for advanced prostate cancer.

Therapeutic use of individual cytokine-based immunotherapeutic agents has shown modest clinical success, and many current strategies are focused on the use of specific immunotherapeutic agonists. However, these immunotherapeutics that engage individual receptors of innate immune networks such as the toll-like receptor (TLR), are constrained by variable cellular TLR expression and responsiveness to particular TLR agonists, as well as the specific cellular context of different tumors.

DCs, antigen-presenting cells (APCs) that play key roles in linking the innate immunity with adaptive immune responses, are present in low numbers in all body tissues and are specialized in the uptake, transport, processing, and presentation of antigens to T cells. DCs pulsed with tumor lysates in vitro enhance therapeutic antitumor immune responses after vaccination. Due to the ability of tumor cells to suppress patient's antitumor immune response, DC-based immunotherapeutic options need to undergo major refinements before can be used to treat metastatic tumors.

Photodynamic therapy (PDT), an anti-tumor therapeutic modality that has approval for the treatment of oncological diseases in a number of countries including the US, involves administration of a photosensitizer (PS) followed by illumination of the tumor with a long wavelength (600-800 nm) light producing reactive oxygen species (ROS) resulting vascular shutdown, cancer cell apoptosis, and the induction of a host immune response. Possible speculations of the mechanism of PDT-induced immune activation include alterations of the tumor microenvironment by stimulating proinflammatory cytokines and direct effects of PDT on the tumor that increases immunogenicity. PDT can increase DC maturation and differentiation, which leads to generation of tumor specific cytotoxic CD8 T cells, which can destroy distant deposits of untreated tumor.

Zinc phthalocyanins (ZnPcs) are a class of long wavelength absorbing PS which are known to successfully address the drawbacks exhibited by the FDA approved PDT drug Photofrin. ZnPc-based sensitizers significantly inhibit the inner mitochondrial membrane enzymes cytochrome c oxidase and F(0)F(1) ATP synthase and upon photoactivation initiates an important cell-death pathway involving the release of cytochrome c from mitochondria to the cytoplasm, thereby triggering caspase activation, and initiation of apoptosis. Preclinical and clinical techniques of PDT are still being optimized to address the major issues that PDT sometimes fails to eradicate the targeted tumor.

We propose that failures of prior studies, in part, may arise from inhomogeneous delivery of the PS within the tumor and in particular it's target organelle, mitochondria of cells, and the inability to produce short-lived singlet oxygen in the mitochondria of tumor cells. We conjecture that exposing DCs in vitro to tumor cell lysates treated with mitochondria-targeted PDT may show different immune response profiles and might unravel the unknown pathways which can be used to improve DC immunotherapy against tumors by enhancing their function.

### SUMMARY

The present disclosure describes, among other things, agents, such as nanoparticles, configured to enhance immunogenicity of cancer cells, such as breast cancer cells. The agents include a mitochondrial targeting moiety and a photosensitizer configured to produce a reactive oxygen species when exposed to light of an appropriate wavelength. Activated cancer cells, which are cancer cells that have been contacted with the nanoparticle and exposed to the appropriate wavelength of light, or supernatants thereof, may be used to generate dendritic cells ex vivo or to cause dendritic cells to produce interferon-gamma (IFN-gamma). The production of INF-gamma by dendritic cells is surprising, as IFN-gamma is typically produced by T-cells. As described herein, the production of INF-gamma by dendritic cells is greater when using agents that target the photosensitizer to mitochondria of the cancer cells relative to untargeted nanoparticles or free photosensitizer.

In embodiments, an agent includes a mitochondrial targeting moiety; and photosensitizer configured to produce a reactive oxygen species when illuminated with light having a particular wavelength. The agent can be a nanoparticle. The nanoparticle, in embodiments, includes a hydrophobic nanoparticle core and a hydrophilic layer surrounding the core.

In embodiments, a nanoparticle includes a hydrophobic nanoparticle core; a hydrophilic layer surrounding the core; and photosensitizer configured to produce a reactive oxygen species when illuminated with light having a particular wavelength.

In embodiments, a nanoparticle includes photosensitizer configured to produce a reactive oxygen species when illuminated with light having a particular wavelength.

In embodiments, a method for treating cancer in a patient in need thereof, includes contacting a cancer cell with an agent that comprises a photosensitizer. The agent can be a nanoparticle.

In embodiments, a method for enhancing the immunogenicity of a cancer cell, comprises contacting the cancer cell with a agent that comprises a photosensitizer. The agent can be a nanoparticle.

In embodiments, a method for activating a dendritic cell includes contacting the dendritic cell with a cancer cell, or a supernatant thereof, where the cancer cell has been contacted with a photosensitizer and exposed to light of a wavelength configured to cause the photosensitizer to produce a reactive oxygen species. The photosensitizer may be present in a nanoparticle.

In embodiments, a method for producing INF-gamma from a dendritic cell includes contacting the dendritic cell with a cancer cell, or a supernatant thereof, where the cancer cell has been contacted with a photosensitizer and exposed to light of a wavelength configured to cause the photosensitizer to produce a reactive oxygen species. The photosensitizer may be present in a nanoparticle.

Advantages of one or more of the various embodiments presented herein over prior nanoparticles, imaging methodologies, treatment modalities, or the like will be readily apparent to those of skill in the art based on the following detailed description when read in conjunction with the accompanying drawings.

### BRIEF DESCRIPTION OF THE DRAWINGS

FIG. 1 is schematic drawing illustrating a reaction scheme for the synthesis of a ZnPc-loaded mitochondrial-targeted nanoparticle, and proposed mechanism of action showing activation by a 660 nm laser inside mitochondria to produce ROS which caused cell death via apoptosis and necrosis.

FIG. 2: Characterization of mitochondria-targeted and non-targeted NPs for light triggered immune activation. Size, zeta potential, and ZnPc loading in (A) targeted and (B) non-targeted NPs. (C) TEM images of targeted (T) and non-targeted (NT) empty and ZnPc-loaded NPs.

FIG. 3: NP construction for light triggered immune activation. (A) HeLa, HL-60, MCF-7 cancer cells respond differently to light activation compared to mesenchymal stem cells. (B) FACS analysis using Annexin V-FITC/PI staining for apoptosis detection in MCF-7 cells on treatment with T-ZnPc-NP, NT-ZnPc-NP, and free ZnPc in the dark or irradiation with a 660 nm LASER for 1 min.

FIG. 4: Mitochondria-targeted light triggered immune activation.

FIG. 5: DC maturation assay. MCF-7 cells stimulated with T-ZnPc-NP induces expression of CD11c, CD86, and CD40 markers on DCs.

FIG. 6: CD8 T cell activation assay. DCs exposed to lysates of MCF-7 cells treated with different conjugates with or without subjected to PDT with a 660 nm LASER (20 mW) for 1 min activates CD8+ T-cells.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

Agents described herein include one or more mitochondrial targeting moieties and one or more photosensitizer. The photosensitizer is configured to generate a reactive oxygen species when exposed to a predetermined wavelength of light. The agents may further include one or more cancer targeting moiety. In embodiments, the agents are nanoparticles.

Nanoparticles, as described herein, include, in embodiments, a hydrophobic core, a hydrophilic layer surrounding the core, and one or more mitochondrial targeting moieties, as well as one or more photosensitizer. The nanoparticle may further include one or more cancer targeting moieties. The photosensitizers are preferably released from the nanoparticle at a desired rate. In embodiments, the core is biodegradable and releases the photosensitizers as the core is degraded or eroded. The targeting moieties preferably extend outwardly from the core so that they are available for interaction with cellular components or so that they affect surface properties of the nanoparticle, which interactions or surface properties will favor preferential distribution to mitochondria. The targeting moieties may be tethered to the core or components that interact with the core.

Nanoparticles having a mitochondrial targeting moiety and a photosensitizer may be made in any suitable manner. In embodiments, nanoparticles can be constructed as described in (i) copending US provisional application, filed on Feb. 13, 2012, naming Shanta Dhar as an inventor, and describing information generally as disclosed in Marrache and Dhar (2012, Oct. 2, 2012), Proc. Natl. Acad. Sci. USA, vol. 109 (40), pages 16288-16293; and (ii) PCT patent application no. PCT/US2012/053307, filed on Aug. 31, 2012, which claims priority to U.S. Provisional Patent Application No. 61/529,637 filed on Sep. 9, 2012, each of which patent applications are incorporated herein by reference in their respective entireties to the extent that they do not conflict with the present disclosure.

### I. CORE

The core of a nanoparticle may be formed from any suitable component or components. Preferably, the core is formed from hydrophobic components such as hydrophobic polymers or hydrophobic portions of polymers. The core may also or alternatively include block copolymers that have hydrophobic portions and hydrophilic portions that may self-assemble in an aqueous environment into particles having the hydrophobic core and a hydrophilic outer surface. In embodiments, the core comprises one or more biodegradable polymer or a polymer having a biodegradable portion.

Any suitable synthetic or natural bioabsorbable polymers may be used. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable.

In various embodiments, described herein the core comprises PLGA. PLGA is a well-known and well-studied hydrophobic biodegradable polymer used for the delivery and release of therapeutic agents at desired rates.

Preferably, the at least some of the polymers used to form the core are amphiphilic having hydrophobic portions and hydrophilic portions. The hydrophobic portions can form the core, while the hydrophilic regions may form a layer surrounding the core to help the nanoparticle evade recognition by the immune system and enhance circulation half-life. Examples of amphiphilic polymers include block copolymers having a hydrophobic block and a hydrophilic block. In embodiments, the core is formed from hydrophobic portions of a block copolymer, a hydrophobic polymer, or combinations thereof.

The ratio of hydrophobic polymer to amphiphilic polymer may be varied to vary the size of the nanoparticle. In embodiments, a greater ratio of hydrophobic polymer to amphiphilic polymer results in a nanoparticle having a larger diameter. Any suitable ratio of hydrophobic polymer to amphiphilic polymer may be used. In embodiments, the nanoparticle includes about a 50/50 ratio by weight of amphiphilic polymer to hydrophobic polymer or ratio that includes more amphiphilic polymer than hydrophilic polymer, such as about a 20/80 ratio, about a 30/70 ratio, about a 20/80 ratio, about a 55/45 ratio, about a 60/40 ratio, about a 65/45 ratio, about a 70/30 ratio, about a 75/35 ratio, about a 80/20 ratio, about a 85/15 ratio, about a 90/10 ratio, about a 95/5 ratio, about a 99/1 ratio, or about 100% amphiphilic polymer.

In embodiments, the hydrophobic polymer comprises PLGA, such as PLGA-COOH or PLGA-OH or PLGA-TPP. In embodiments, the amphiphilic polymer comprises PLGA and PEG, such as PLGA-PEG. The amphiphilic polymer may be a dendritic polymer having branched hydrophilic portions. Branched polymers may allow for attachment of more than moiety to terminal ends of the branched hydrophilic polymer tails, as the branched polymers have more than one terminal end.

Nanoparticles having a diameter of about 250 nm or less are generally more effectively targeted to mitochondria than nanoparticles having a diameter of greater than about 250 nm. In embodiments, a nanoparticle effective for mitochondrial targeting has a diameter of about 200 nm or less, 190 nm or less, about 180 nm or less, about 170 nm or less, about 160 nm or less, about 150 nm or less, about 140 nm or less, about 130 nm or less, about 120 nm or less, about 110 nm or less, about 100 nm or less, about 90 nm or less, about 80 nm or less, about 80 nm or less, about 80 nm or less, about 80 nm or less, about 80 nm or less, about 70 nm or less, about 60 nm or less, about 50 nm or less, about 40 nm or less, about 30 nm or less, about 20 nm or less, or about 10 nm or less. In embodiments, a nanoparticle has a diameter of from about 10 nm to about 250 nm, such as from about 20 nm to about 200 nm, from about 50 nm to about 160 nm, from about 60 nm to about 150 nm, from about 70 nm to about 130 nm, from about 80 nm to about 120 nm, from about 80 nm to about 100 nm, or the like.

### II. HYDROPHILIC LAYER SURROUNDING THE CORE

The nanoparticles described herein may optionally include a hydrophilic layer surrounding the hydrophilic core. The hydrophilic layer may assist the nanoparticle in evading recognition by the immune system and may enhance circulation half-life of the nanoparticle.

As indicated above, the hydrophilic layer may be formed, in whole or in part, by a hydrophilic portion of an amphiphilic polymer, such as a block co-polymer having a hydrophobic block and a hydrophilic block.

Any suitable hydrophilic polymer or hydrophilic portion of an amphiphilic polymer may form the hydrophilic layer or portion thereof. The hydrophilic polymer or hydrophilic portion of a polymer may be a linear or dendritic polymer. Examples of suitable hydrophilic polymers include polysaccharides, dextran, chitosan, hyaluronic acid, polyethylene glycol, polymethylene oxide, and the like.

In embodiments, a hydrophilic portion of a block copolymer comprises polyethylene glycol (PEG). In embodiments, a block copolymer comprises a hydrophobic portion comprising PLGA and a hydrophilic portion comprising PEG.

A hydrophilic polymer or hydrophilic portion of a polymer may contain moieties that are charged under physiological conditions, which may be approximated by a buffered saline solution, such as a phosphate or citrate buffered saline solution, at a pH of about 7.4, or the like. Such moieties may contribute to the charge density or zeta potential of the nanoparticle. Zeta potential is a term for electrokinetic potential in colloidal systems. While zeta potential is not directly measurable, it can be experimentally determined using electrophoretic mobility, dynamic electrophoretic mobility, or the like.

It has been found that zeta potential may play an important role in the ability of nanoparticles to accumulate in mitochondria, with higher zeta potentials generally resulting in increased accumulation in the mitochondria. In embodiments, the nanoparticles have a zeta potential, as measured by dynamic light scattering, of about 0 mV or greater. For example, a nanoparticle may have a zeta potential of about 1 mV or greater, of about 5 mV or greater, of about 7 mV or greater, or about 10 mV or greater, or about 15 mV or greater, of about 20 mV or greater, about 25 mV or greater, about 30 mV or greater, about 34 mV or greater, about 35 mV or greater, or the like. In embodiments, a nanoparticle has a zeta potential of from about 0 mV to about 100 mV, such as from about 1 mV to 50 mV, from about 2 mV to about 40 mV, from about 7 mV to about 35 mV, or the like.

Any suitable moiety that may be charged under physiological conditions may be a part of or attached to a hydrophilic polymer or hydrophilic portion of a polymer. In embodiments, the moiety is present at a terminal end of the polymer or hydrophilic portion of the polymer. Of course, the moiety may be directly or indirectly bound to the polymer backbone at a location other than at a terminal end. Due to the substantial negative electrochemical potential maintained across the inner mitochondrial membrane, cations, particularly if delocalized, are effective at crossing the hydrophobic membranes and accumulating in the mitochondrial matrix. Cationic moieties that are known to facilitate mitochondrial targeting are discussed in more detail below. However, cationic moieties that are not particularly effective for selective mitochondrial targeting may be included in nanoparticles or be bound to hydrophilic polymers or portions of polymers. In embodiments, anionic moieties may form a part of or be attached to the hydrophilic polymer or portion of a polymer. The anionic moieties or polymers containing the anionic moieties may be included in nanoparticles to tune the zeta potential, as desired. In embodiments, a hydrophilic polymer or portion of a polymer includes a hydroxyl group that can result in an oxygen anion when placed in a physiological aqueous environment. In embodiments, the polymer comprises PEG-OH where the OH serves as the charged moiety under physiological conditions.

### III. MITOCHONDRIA TARGETING MOIETIES

The nanoparticles described herein include one or more moieties that target the nanoparticles to mitochondria. As used herein, “targeting” a nanoparticle to mitochondria means that the nanoparticle accumulates in mitochondria relative to other organelles or cytoplasm at a greater concentration than substantially similar non-targeted nanoparticle. A substantially similar non-target nanoparticle includes the same components in substantially the same relative concentration (e.g., within about 5%) as the targeted nanoparticle, but lacks a targeting moiety.

The mitochondrial targeting moieties may be tethered to the core in any suitable manner, such as binding to a molecule that forms part of the core or to a molecule that is bound to the core. In embodiments, a targeting moiety is bound to a hydrophilic polymer that is bound to a hydrophobic polymer that forms part of the core. In embodiments, a targeting moiety is bound to a hydrophilic portion of a block copolymer having a hydrophobic block that forms part of the core.

The targeting moieties may be bound to any suitable portion of a polymer. In embodiments, the targeting moieties are attached to a terminal end of a polymer. In embodiments, the targeting moieties are bound to the backbone of the polymer, or a molecule attached to the backbone, at a location other than a terminal end of the polymer. More than one targeting moiety may be bound to a given polymer. In embodiments, the polymer is a dendritic polymer having multiple terminal ends and the targeting moieties may be bound to more than one of terminal ends.

The polymers, or portions thereof, to which the targeting moieties are bound may contain, or be modified to contain, appropriate functional groups, such as —OH, —COOH, —NH₂, —SH, —N₃, —Br, —Cl, —I, or the like, for reaction with and binding to the targeting moieties that have, or are modified to have, suitable functional groups.

Examples of targeting moieties tethered to polymers presented throughout this disclosure for purpose of illustrating the types of reactions and tethering that may occur. However, one of skill in the art will understand that tethering of targeting moieties to polymers may be carried out according to any of a number of known chemical reaction processes.

Targeting moieties may be present in the nanoparticles at any suitable concentration. In embodiments, the concentration may readily be varied based on initial in vitro analysis to optimize prior to in vivo study or use. In embodiments, the targeting moieties will have surface coverage of from about 5% to about 100%.

Any suitable moiety for facilitating accumulation of the nanoparticle within the mitochondrial matrix may be employed. Due to the substantial negative electrochemical potential maintained across the inner mitochondrial membrane, delocalized lipophilic cations are effective at crossing the hydrophobic membranes and accumulating in the mitochondrial matrix. Triphenyl phosophonium (TPP) containing compounds can accumulate greater than 1000 fold within the mitochondrial matrix. Any suitable TPP-containing compound may be used as a mitochondrial matrix targeting moiety. Representative examples of TPP-based moieties may have structures indicated below in Formula I, Formula II or Formula III:

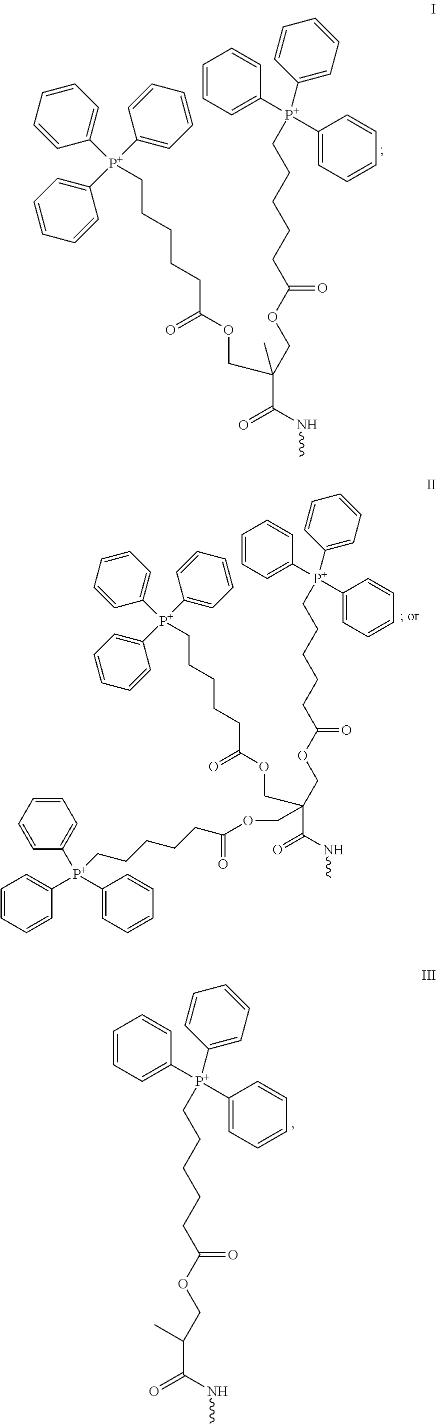

where the amine (as depicted) may be conjugated to a polymer or other component for incorporation into the nanoparticle.

In embodiments, the delocalized lipophilic cation for targeting the mitochondrial matrix is a rhodamine cation, such as Rhodamine 123 having Formula IV as depicted below:

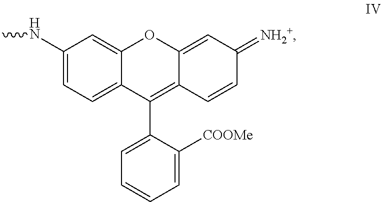

where the secondary amine (as depicted) may be conjugated to a polymer, lipid, or the like for incorporation into the nanoparticle.

Of course, non-cationic compounds may serve to target and accumulate in the mitochondrial matrix. By way of example, Szeto-Shiller peptide may serve to target and accumulate a nanoparticle in the mitochondrial matrix. Any suitable Szeto-Shiller peptide may be employed as a mitochondrial matrix targeting moiety. Non-limiting examples of suitable Szeto-Shiller peptides include SS-02 and SS-31, having Formula V and Formula VI, respectively, as depicted below:

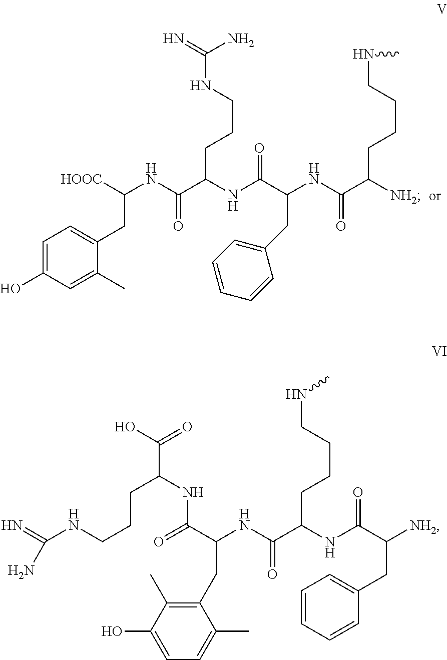

where the secondary amine (as depicted) may be conjugated to a polymer, lipid, or the like for incorporation into the nanoparticle.

For purposes of example, a reaction scheme for synthesis of PLGA-PEG-TPP is shown below in Scheme I. It will be understood that other schemes may be employed to synthesize PLGA-PEG-TPP and that similar reaction schemes may be employed to tether other mitochondrial targeting moieties to PLGA-PEG or to tether moieties to other polymer or components of a nanoparticle.

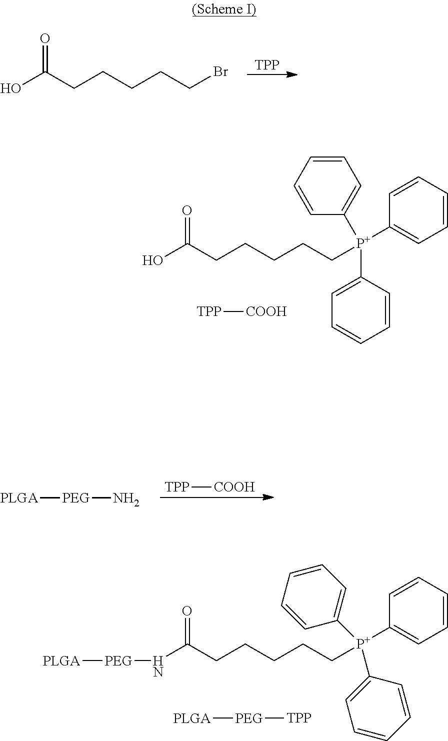

Preferably, a targeting moiety is attached to a hydrophilic polymer or hydrophilic portion of a polymer so that the targeting moiety will extend from the core of the nanoparticle to facilitate the effect of the targeting moiety.

It will be understood that the mitochondrial targeting moiety may alter the zeta potential of a nanoparticle. Accordingly, the zeta potential of a nanoparticle may be tuned by adjusting the amount of targeting moiety included in the nanoparticle. The zeta potential may also be adjusted by including other charged moieties, such as charged moieties of, or attached to, hydrophilic polymers or hydrophilic portions of polymers.

In embodiments, charged moieties are provided only by, or substantially by, mitochondrial targeting moieties. In embodiments, about 95% or more of the charged moieties are provided by mitochondrial targeting moieties. In embodiments, about 90% or more of the charged moieties are provided by mitochondrial targeting moieties. In embodiments, about 85% or more of the charged moieties are provided by mitochondrial targeting moieties. In embodiments, about 80% or more of the charged moieties are provided by mitochondrial targeting moieties. In embodiments, about 75% or more of the charged moieties are provided by mitochondrial targeting moieties. In embodiments, about 70% or more of the charged moieties are provided by mitochondrial targeting moieties. In embodiments, about 65% or more of the charged moieties are provided by mitochondrial targeting moieties. In embodiments, about 60% or more of the charged moieties are provided by mitochondrial targeting moieties. In embodiments, about 55% or more of the charged moieties are provided by mitochondrial targeting moieties. In embodiments, about 50% or more of the charged moieties are provided by mitochondrial targeting moieties. Of course, the mitochondrial targeting moieties may provide any suitable amount or percentage of the charged moieties.

In embodiments, the nanoparticles are formed by blending a polymer that include a mitochondrial targeting moiety with a polymer that includes a charged moiety other than a mitochondrial targeting moiety.

### IV. PHOTOSENSITIZER

A nanoparticle, as described herein, may include any one or more photosensitizer. As used herein a photosensitizer is compound that, when exposed to light of a predetermined wavelength generates a reactive oxygen species. A reactive oxygen species is a chemically reactive molecule containing oxygen. Examples of reactive oxygen species are molecules that include oxygen ions, oxygen radicals, peroxides, and the like. The photosensitizer may be embedded in, or contained within, the core of the nanoparticle. Preferably, the photosensitizer is released from the core at a desired rate. If the core is formed from a polymer (such as PLGA) or combination of polymers having known release rates, the release rate can be readily controlled.

In embodiments, a photosensitizer or precursor thereof is conjugated to a polymer, or other component of a nanoparticle, in a manner described above with regard to targeting moieties. The photosensitizer may be conjugated via a cleavable linker so that the photosensitizer may be released when the nanoparticle reaches the target location, such as mitochondria.

The photosensitizer may be present in the nanoparticle at any suitable concentration. For example, a photosensitizer may be present in the nanoparticle at a concentration from about 0.0001% to about 40% by weight of the nanoparticle.

Any suitable photosensitizer may be used. Examples of photosensitizers include porphyrins, chlorophylls and dyes. For example, aminolevullinic acid (ALA), silicon phthalocyanine PC 4, m-tetrahydroxyphenylchlorin (mTHPC) and mono L-aspartyl chlorin e6 (NPe6) are photosensitizers that may be used. Examples of commercially available photosensitizers include_Allumera, Photofrin, Visudyne, Levulan, Foscan, Metvix, Hexvix, Cysview, and Laserphyrin. Examples of photosensitizer in development include Antrin, Photochlor, Photosens, Photrex, Lumacan, Cevira, Visonac, BF-200 ALA, Amphinex, and Azadipyrromethenes.

In embodiments, the photosensitizer is a pthalocyanin, such as a zinc pthalocyanin.

In embodiments, the photosensitizer is configured to produce a reactive oxygen species when exposed to light in a wavelength of about 600 nm to about 800 nm. Of course, any suitable wavelength of light may be used, depending on the photosensitizer employed.

### V. CONTRAST AGENTS

A nanoparticle as described herein may include one or more contrast agents for purpose of imaging, visualization or diagnosis. In embodiments, imaging is performed to verifying that therapeutic nanoparticles are being properly trafficked to mitochondria. Any suitable contrast agent may be employed. In embodiments, the contrast agent is suitable for in vivo magnetic resonance imaging (MRI), such as iron oxide (10) nanocrystals or gadolimium complexes. In embodiments, the contrast agent is suitable for ex vivo/in vivo optical imaging, such as quantum dot (QD) (fluorescence) or fluorescent dyes, cdots, pdots, or the like. In embodiments, the nanoparticle includes both contrast agents for MRI and agents for fluorescent optical imaging.

Contrast agents may be incorporated into the nanoparticle in any suitable manner. In embodiments, the contrast agents are incorporated into the core or are contained within the core. In embodiments, the contrast agents are tethered to a polymer or other component of the nanoparticle. Such tethering can be carried out as described above with regard to other components of the nanoparticle, such as targeting moieties.

Contrast agents may be present in a nanoparticle in any suitable amount. In embodiments, a contrast agent is present in a nanoparticle from about 0.05% by weight to about 30% by weight of the nanoparticle.

### VI. CANCER TARGETING MOIETY

In embodiments, a nanoparticle described herein may include a cancer targeting moiety. Such moieties include moieties that preferentially or selectively interact with molecules or portions thereof that are present on the cell surface of cancer cells. Preferably the molecules or portions thereof that are presented on the surface of the cancer cells are present at a concentration greater than non-cancer cells. Such cancer targeting moieties are well known to those of skill in the art.

Cancer targeting moieties may be bound to the nanoparticle in any suitable manner, such as in manner similar to those described above with regard to mitochondrial targeting moieties.

In embodiments, a cancer targeting moiety comprises an antibody or a fragment or a portion of an antibody. In embodiments, a cancer targeting moiety is a receptor agonist or antagonist.

In embodiments, a cancer targeting moiety selectively binds to a growth factor receptor.

Non-limiting examples of molecules to which cancer targeting moieties may bind include human epidermal growth factor receptor 2 (HER2), epidermal growth factor receptor (EGFR), beta lymphocyte antigen CD20 (CD20), vascular endothelial growth factor receptor (VEGFR), and platelet derived growth factor receptor (PDGFR).

### VII. SYNTHESIS OF NANOPARTICLE

Nanoparticles, as described herein, may be synthesized or assembled via any suitable process. Preferably, the nanoparticles are assembled in a single step to minimize process variation. A single step process may include nanoprecipitation and self-assembly.

In general, the nanoparticles may be synthesized or assembled by dissolving or suspending hydrophobic components in an organic solvent, preferably a solvent that is miscible in an aqueous solvent used for precipitation. In embodiments, acetonitrile is used as the organic solvent, but any suitable solvent (such as DMF, DMSO, acetone, or the like) may be used. Hydrophilic components are dissolved in a suitable aqueous solvent, such as water, 4 wt-% ethanol, or the like. The organic phase solution may be added drop wise to the aqueous phase solution to nanoprecipitate the hydrophobic components and allow self-assembly of the nanoparticle in the aqueous solvent.

A process for determining appropriate conditions for forming the nanoparticles may be as follows. Briefly, functionalized polymers and other components, if included or as appropriate, may be co-dissolved in organic solvent mixtures. This solution may be added drop wise into hot (e.g, 65° C.) aqueous solvent (e.g, water, 4 wt-% ethanol, etc.), whereupon the solvents will evaporate, producing nanoparticles with a hydrophobic core surrounded by a hydrophilic polymer component, such as PEG. Once a set of conditions where a high (e.g., >75%) level of targeting moiety surface loading has been achieved, contrast agents or therapeutic agents may be included in the nanoprecipitation and self-assembly of the nanoparticles.

If results are not desirably reproducible by manual mixing, microfluidic channels may be used.

Nanoparticles may be characterized for their size, charge, stability, IO and QD loading, drug loading, drug release kinetics, surface morphology, and stability using well-known or published methods.

Nanoparticle properties may be controlled by (a) controlling the composition of the polymer solution, and (b) controlling mixing conditions such as mixing time, temperature, and ratio of water to organic solvent. The likelihood of variation in nanoparticle properties increases with the number of processing steps required for synthesis.

The size of the nanoparticle produced can be varied by altering the ratio of hydrophobic core components to amphiphilic shell components. Nanoparticle size can also be controlled by changing the polymer length, by changing the mixing time, and by adjusting the ratio of organic to the phase. Prior experience with nanoparticles from PLGA-b-PEG of different lengths suggests that nanoparticle size will increase from a minimum of about 20 nm for short polymers (e.g. PLGA₃₀₀₀-PEG₇₅₀) to a maximum of about 150 nm for long polymers (e.g. PLGA_{100,000}-PEG_{10,000}). Thus, molecular weight of the polymer will serve to adjust the size.

Nanoparticle surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers.

Once formed, the nanoparticles may be collected and washed via centrifugation, centrifugal ultrafiltration, or the like. If aggregation occurs, nanoparticles can be purified by dialysis, can be purified by longer centrifugation at slower speeds, can be purified with the use surfactant, or the like.

Once collected, any remaining solvent may be removed and the particles may be dried, which should aid in minimizing any premature breakdown or release of components. The nanoparticles may be freeze dried with the use of bulking agents such as mannitol, or otherwise prepared for storage prior to use.

It will be understood that therapeutic agents may be placed in the organic phase or aqueous phase according to their solubility.

Nanoparticles described herein may include any other suitable components, such as phospholipids or cholesterol components, generally know or understood in the art as being suitable for inclusion in nanoparticles. Copending patent application, PCT/US2012/053307, describes a number of additional components that may be included in nanoparticles.

Nanoparticles disclosed in PCT/US2012/053307 include targeting moieties that target the nanoparticles to apoptotic cells, such as moieties that target phosphatidylserine (PS). The targeting moieties are conjugated to a component of the nanoparticle. Such moieties include various polypeptides or zinc 2,2′-dipicolylamine (Zn²⁺-DPA) coordination complexes. In embodiments, the nanoparticles described herein are free or substantially fee of apoptotic cell targeting moieties. In embodiments, the nanoparticles described herein are free or substantially fee of apoptotic cell targeting moieties that are conjugated to a component of the nanoparticle. In embodiments, the nanoparticles described herein are free or substantially fee of PS targeting moieties. In embodiments, the nanoparticles described herein are free or substantially fee of PS targeting moieties that are conjugated to a component of the nanoparticle. In embodiments, the nanoparticles described herein are free or substantially fee of PS-polypeptide targeting moieties or Zn²⁺-DPA moieties. In embodiments, the nanoparticles described herein are free or substantially fee of PS-polypeptide targeting moieties or Zn²⁺-DPA moieties that are conjugated to a component of the nanoparticle.

Nanoparticles disclosed in PCT/US2012/053307 include macrophage targeting moieties, such as simple sugars, conjugated to components of the nanoparticles. In embodiments, the nanoparticles described herein are free or substantially free of macrophage targeting moieties. In embodiments, the nanoparticles described herein are free or substantially free of macrophage targeting moieties that are conjugated to the nanoparticle or a component thereof. In embodiments, the nanoparticles described herein are free or substantially free of simple sugar moieties. In embodiments, the nanoparticles described herein are free or substantially free of simple sugar moieties that are conjugated to the nanoparticle or a component thereof.

### VIII. USE AND TESTING

In general, a nanoparticle as described herein may be contacted with a cancer cell to enhance the immunogenicity of the cancer cell following light activation of the photosensitizer. It will be understood that contacting a cancer cell with a photosensitizer that is not included in a nanoparticle is contemplated herein.

Such enhanced immunogenicity cancer cells, or “activated” cancer cells, or supernatants thereof, may be contacted with dendritic cells to activate the dendritic cells or to cause the dendritic cells to produce IFN-gamma.

In embodiments, the cancer cells are breast cancer cells, such as MCF-7 cells, NT-1 cells, or the like.

The performance and characteristics of nanoparticles produced herein may be tested or studied in any suitable manner. By way of example, therapeutic efficacy can be evaluated using cell-based assays. Toxicity, bio-distribution, pharmacokinetics, and efficacy studies can be tested in cells or rodents or other mammals. Zebrafish or other animal models may be employed for combined imaging and therapy studies. Rodents, rabbits, pigs, or the like may be used to evaluate diagnostic or therapeutic potential of nanoparticles. Some additional details of studies that may be performed to evaluate the performance or characteristics of the nanoparticles, which may be used for purposes of optimizing the properties of the nanoparticles are described below. However, one of skill in the art will understand that other assays and procedures may be readily performed.

Uptake and binding characteristics of nanoparticles containing a contrast agent may be evaluated in any suitable cell line, such as RAW 264.7, J774, jurkat, and HUVEGs cells. The immunomodulatory role of nanoparticles may be assayed by determining the release of cytokines when these cells are exposed to varying concentrations of nanoparticles. Complement activation may be studied to identify which pathways are triggered using columns to isolate opsonized nanoparticles; e.g. as described in Salvador-Morales C, Zhang L, Langer R, Farokhzad OC, Immunocompatibility properties of lipid—polymer hybrid nanoparticles with heterogeneous surface functional groups, Biomaterials 30: 2231-2240, (2009). Fluorescence measurements may be carried out using a plate reader, FACS, or the like. Because nanoparticle size is an important factor that determines biodistribution, Nanoparticles may be binned into various sizes (e.g., 20-40, 40-60, 60-80, 80-100, 100-150, and 150-300 nm) and tested according to size.

Any cell type appropriate for a photosensitizer employed in a nanoparticle may be used to evaluate therapeutic efficacy or proper targeting. Assays appropriate for the therapeutic or pharmacologic outcome may be employed, as are generally understood or known in the art.

Biodistribution (bioD) and pharmacokinetic (PK) studies may be carried out in rats or other suitable mammals. For PK and bioD analysis, Sprague Dawley rats may be dosed with QD-labeled, apoptosis-targeting, macrophage-targeting nanoparticles or similar nanoparticles without the targeting groups, through a lateral tail vein injection. The bioD may be followed initially by fluorescence imaging for 1-24 h after injection. Animals may be sacrificed; and brain, heart, intestine, liver, spleen, kidney, muscle, bone, lung, lymph nodes, gut, and skin may be excised, weighed, homogenized, and Cd from QD may be quantified using ICP-MS. Tissue concentration may be expressed as % of injected dose per gram of tissue (% ID/g). Blood half-life may be calculated from blood Cd concentrations at various time points

Therapeutic dosages of nanoparticles effective for human use can be estimated from animal studies according to well-known techniques, such as surface area or weight based scaling.

### IX. DEFINITIONS

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “disease” means a condition of a living being or one or more of its parts that impairs normal functioning. As used herein, the term disease encompasses terms such disease, disorder, condition, dysfunction and the like.

As used herein, “treat” or the like means to cure, prevent, or ameliorate one or more symptom of a disease.

As used herein, a compound that is “hydrophobic” is a compound that is insoluble in water or has solubility in water below 1 milligram/liter.

As used herein a compound that is “hydrophilic” is a compound that is water soluble or has solubility in water above 10 milligram/liter.

As used herein, “bind,” “bound,” or the like means that chemical entities are joined by any suitable type of bond, such as a covalent bond, an ionic bond, a hydrogen bond, van der walls forces, or the like. “Bind,” “bound,” and the like are used interchangeable herein with “attach,” “attached,” and the like.

As used herein, a molecule or moiety “attached” to a core of a nanoparticle may be embedded in the core, contained within the core, attached to a molecule that forms at least a portion of the core, attached to a molecule attached to the core, or directly attached to the core.

As used herein, a “derivative” of a compound is a compound structurally similar to the compound of which it is a derivative. Many derivatives are functional derivatives. That is, the derivatives generally a desired function similar to the compound to which it is a derivative. By way of example, triphenyl phosophonium (TPP) is described herein as a mitochondrial targeting moiety because it can accumulate, or cause a compound or complex (such as a nanoparticle) to which it is bound to accumulate, in the mitochondrial matrix. Accordingly, a functional derivative of TPP is a derivative of TPP that may accumulate, or cause a compound or complex to which it is bound to accumulate, in the mitochondrial matrix in a similar concentration as TPP (e.g., within about a 100 fold concentration range, such as within about a 10 fold concentration range).

In the following, non-limiting examples are presented, which describe various embodiments of representative nanoparticles, methods for producing the nanoparticles, and methods for using the nanoparticles.

### EXAMPLES

We constructed a mitochondria-targeted functionalized polymer from FDA approved polymers poly(lactic-co-glycolic acid) (PLGA), polyethyleneglycol (PEG) and a lipophilic triphenyl phosphonium (TPP) cation for mitochondria-targeted delivery of payloads. Polymeric nanoparticles (NPs) from PLGA-b-PEG-TPP takes the advantage of substantial negative mitochondrial inner membrane potential (Δψm) to deliver cargo in the mitochondria. In this study, by serendipity, we observed treatment of DCs to breast cancer cell lysates with mitochondria-targeted PDT shows significant secretion of interferon gamma (IFN-γ), a potent biological response modifier (BRM). Immune escape mechanism is partly associated with a low lymphocyte/tumor cell ratio and insufficient extension of anti-tumor T cell clones and T cell apoptosis within the tumor. The ability to secret IFN-γ from in vitro DC culture treated with mitochondria-targeted-PDT is an indicative that this system will show significant anti-tumor T cell functionality. To our knowledge, this is the first preclinical report demonstrating that mitochondria-targeted PDT-DCs can be used in vitro to very effective production of IFN-γ and this opens up new opportunities for the future development of therapeutic cancer vaccine. In this technology, we discuss the in vitro studies IFN-γ production pathways from DCs stimulated with mitochondria-targeted-PDT-killed breast tumor cells and discuss markedly increased immunogenicity of the mitochondria-targeted-NP PDT compared to non-targeted-PDT.

Various studies were performed regarding the generation of nanoparticles including a photosensitizer, enhanced immunogenicity of cancer cells contacted with the nanoparticles or photosensitizers and exposed to light of a wavelength configured to cause the photosensitizer to generate a reactive oxygen species, generation or activation of dendritic cells contacted with such cancer cells or supernatants thereof, and generation of INF-gamma from dendritic cells contacted with such cancer cells or supernatants thereof.

Nanoparticles were constructed generally as described in (i) copending US provisional application, filed on Feb. 13, 2012, naming Shanta Dhar as an inventor, and describing information generally as disclosed in Marrache and Dhar (2012, Oct. 2, 2012), Proc. Natl. Acad. Sci. USA, vol. 109 (40), pages 16288-16293; and (ii) PCT patent application no. PCT/US2012/053307, filed on Aug. 31, 2012, which claims priority to U.S. Provisional Patent Application No. 61/529,637 filed on Sep. 9, 2012

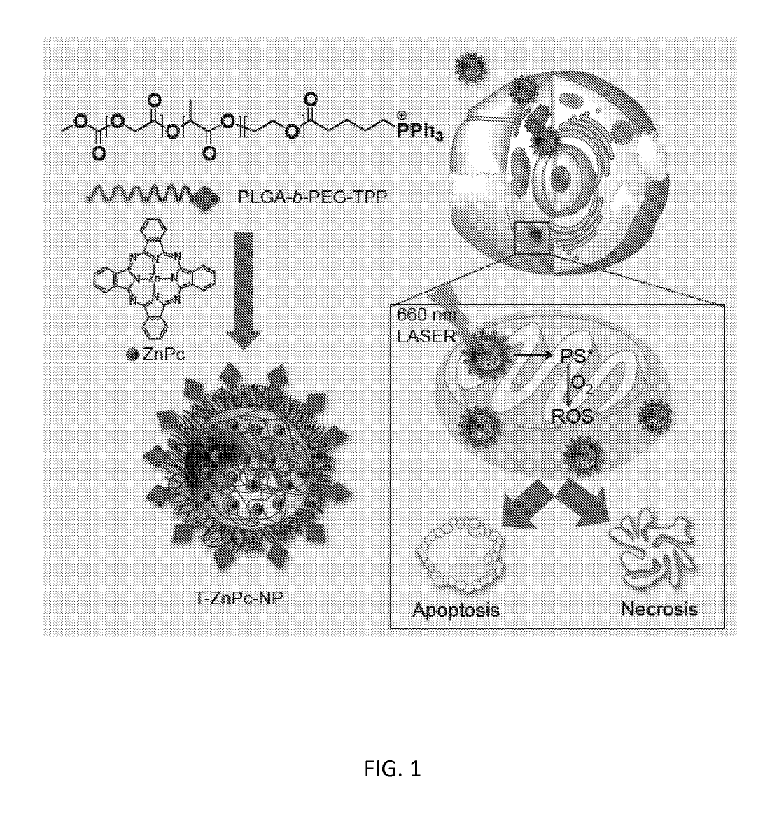

FIG. 1 provides a reaction scheme for the synthesis of a ZnPc-loaded mitochondrial-targeted nanoparticle employed herein. Also depicted in FIG. 1 is a proposed mechanism of action showing activation by a 660 nm laser inside mitochondria to produce ROS which caused cell death via apoptosis and necrosis.

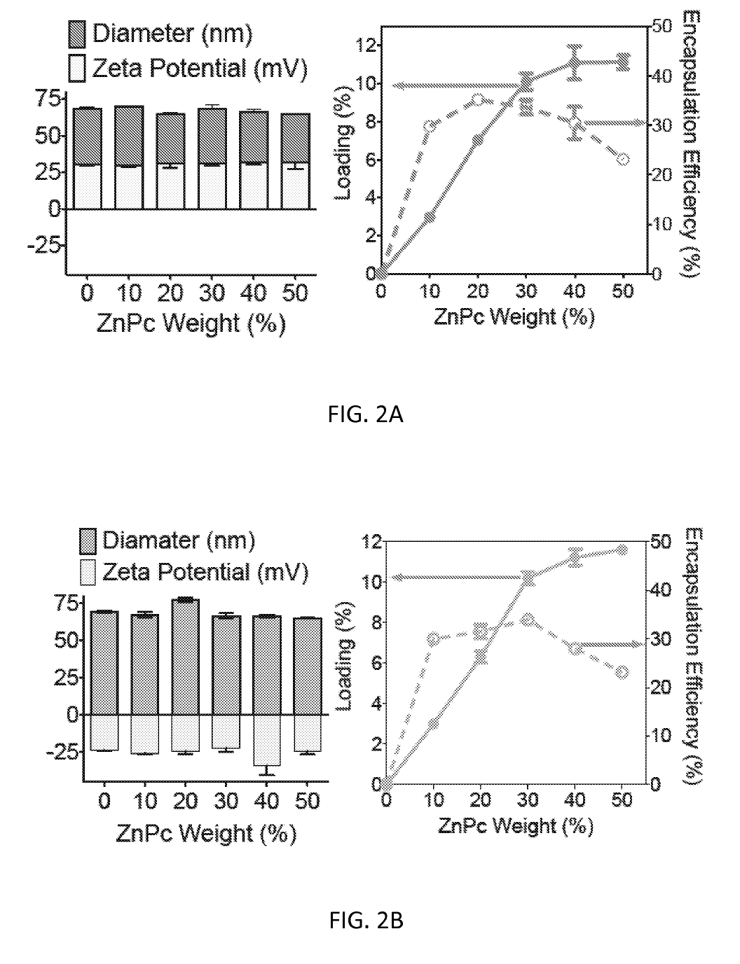

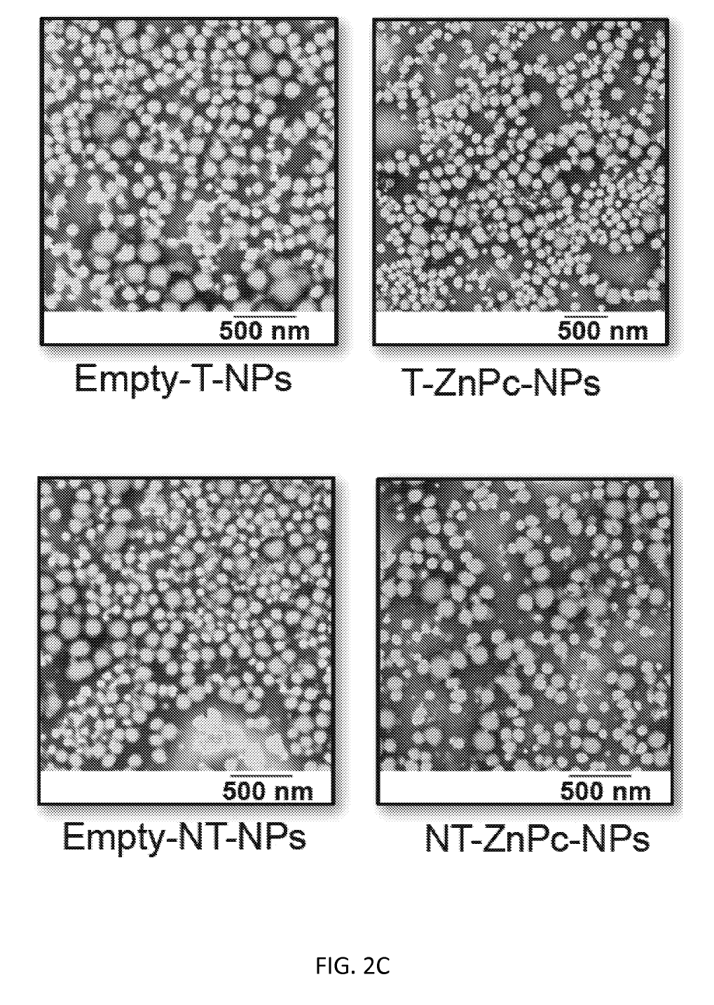

Nanoparticles were characterized generally as described in (i) copending US provisional application, filed on Feb. 13, 2012, naming Shanta Dhar as an inventor, and describing information generally as disclosed in Marrache and Dhar (2012, Oct. 2, 2012), Proc. Natl. Acad. Sci. USA, vol. 109 (40), pages 16288-16293; and (ii) PCT patent application no. PCT/US2012/053307, filed on Aug. 31, 2012, which claims priority to U.S. Provisional Patent Application No. 61/529,637 filed on Sep. 9, 2012. The size, zeta potential and ZnPc loading in targeted and non-targeted NPs are shown in FIG. 2A and FIG. 2B, respectively.

TEM images of targeted (T) and non-targeted (NT) empty and ZnPC-loaded NPs were obtained. TEM samples were negatively stained with sterile 2% (w/v) uranyl acetate solution for 15 min. TEM images are shown in FIG. 2C.

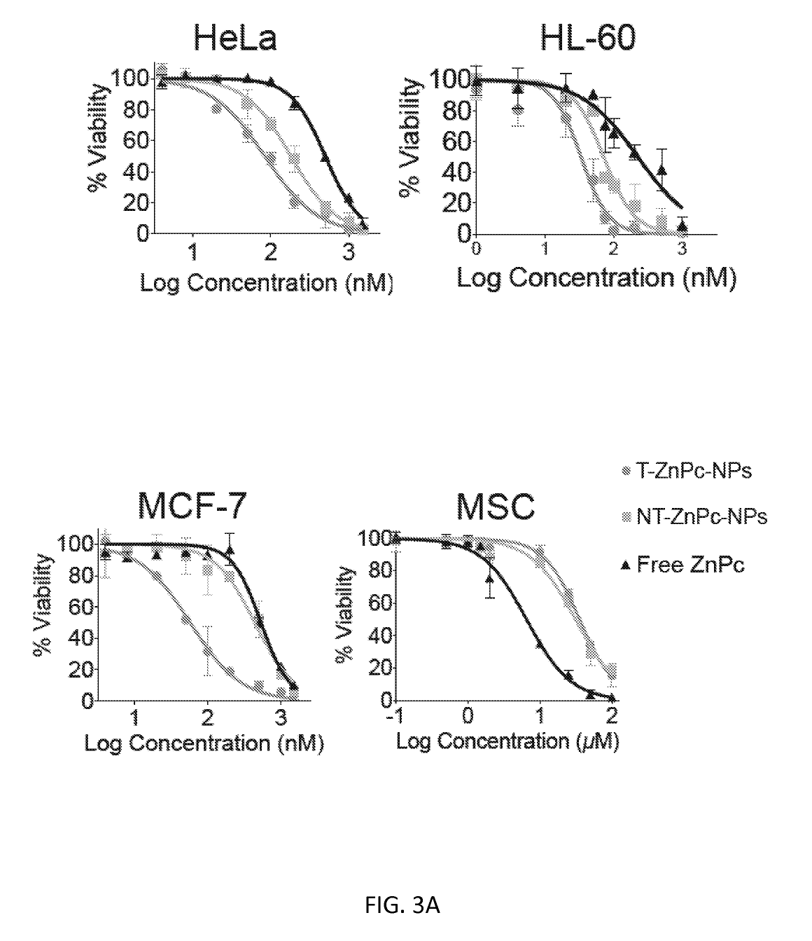

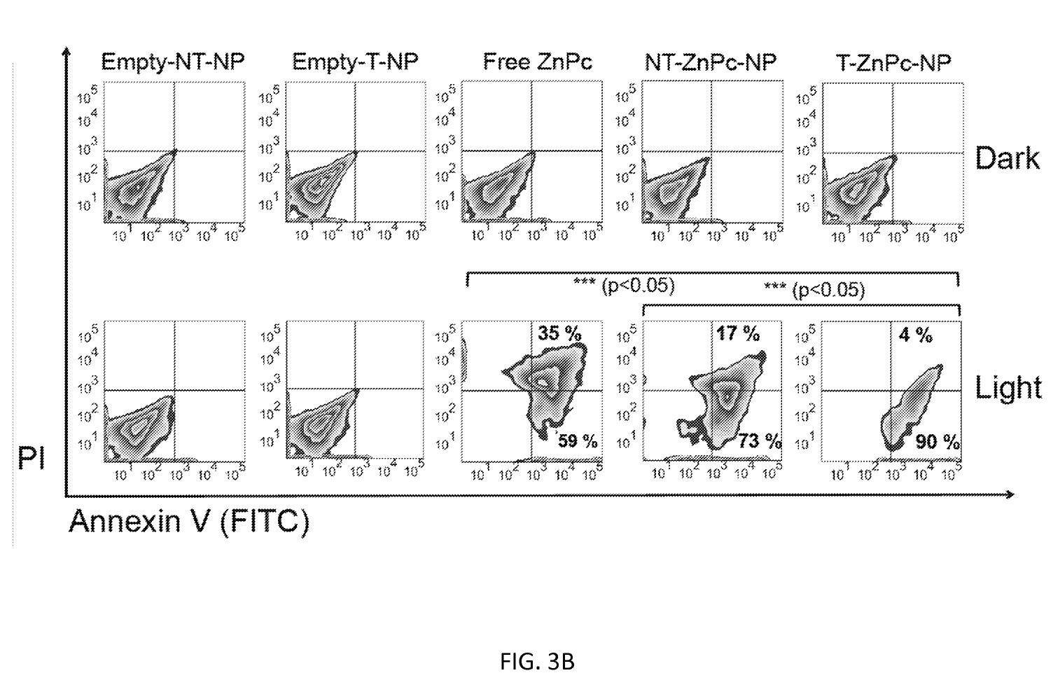

HeLa, HL-60, MCF-7 cancer cells and mesenchymal stem cells were incubated on 96-well plates for 4 h with T-ZnPc-NP, NT-ZnPc-NP, and free ZnPc. Cells were irradiated with a 660 nm LASER (20 mW) for 1 min. Cells were further incubated for additional 72 h and viability was assessed by the MTT assay. Viability results are shown in FIG. 3A, which shows that HeLa, HL-60, MCF-7 cancer cells respond differently to light activation compared to mesenchymal stem cells.

FACS analysis was performed using Annexin V-FITC/PI staining for apoptosis detection in MCF-7 cells on treatment with T-ZnPc-NP, NT-ZnPc-NP, and free ZnPc (concentrations, time, etc.) in the dark or irradiation with a 660 nm LASER for 1 min. Various empty NPs were used as controls. The results are shown in FIG. 3B. Cells in the lower right quadrant indicate Annexin-positive/PI negative, early apoptotic cells. The cells in the upper right quadrant indicate Annexin-positive/PI positive, late apoptotic or necrotic cells. Significant difference value for T-ZnPc-NP from NT-ZnPc-NP and free ZnPc is indicated by *** (p<0.05).

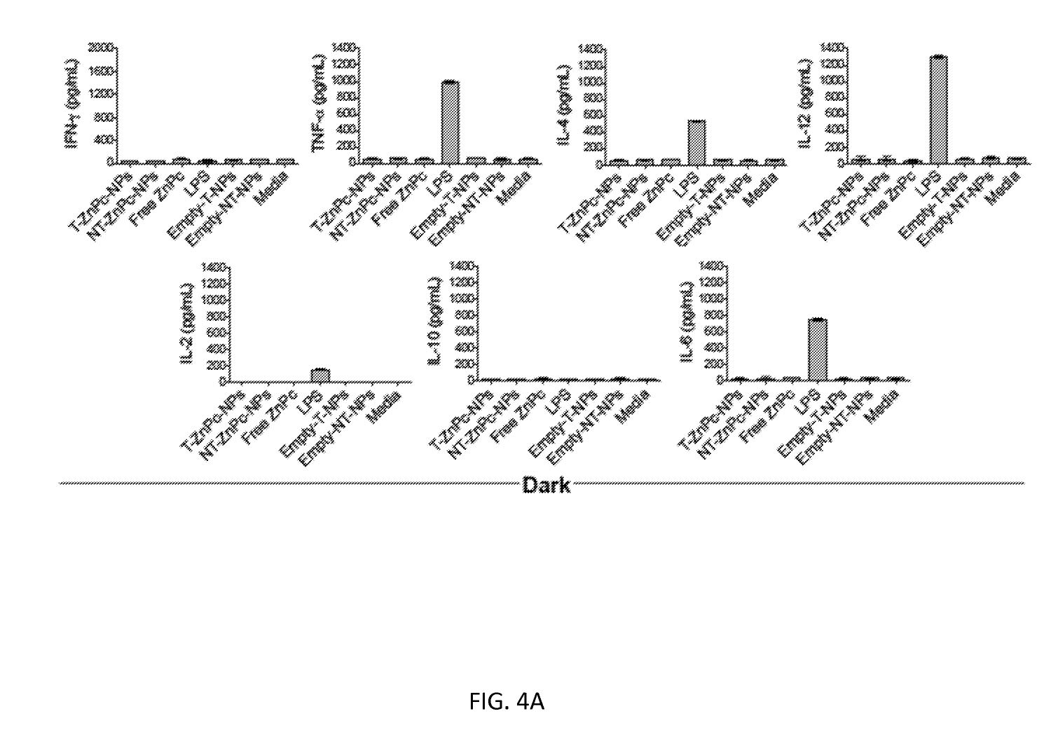

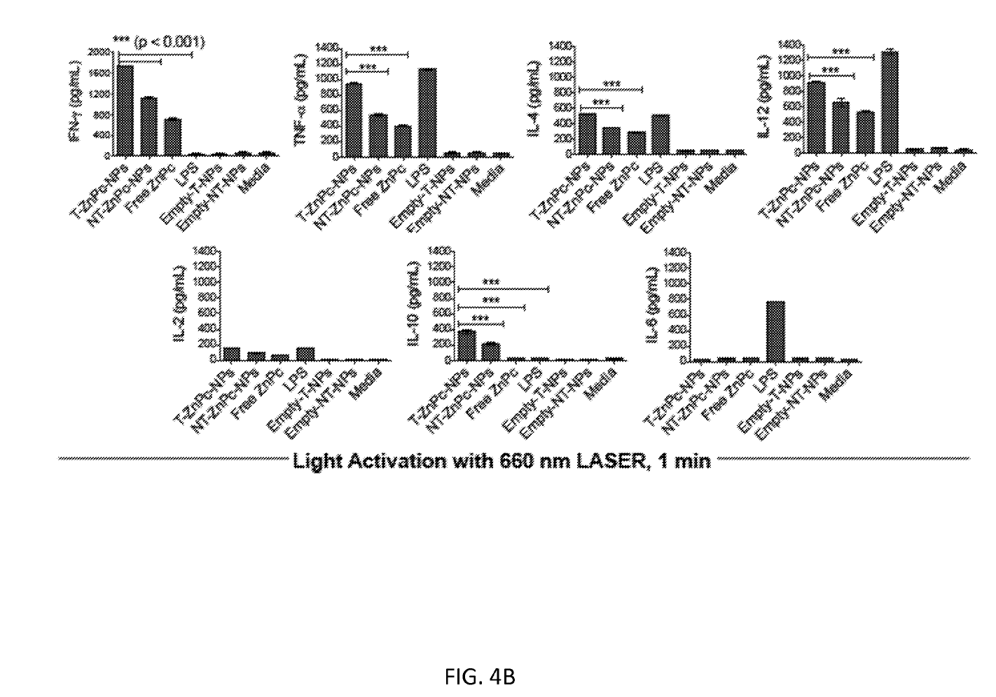

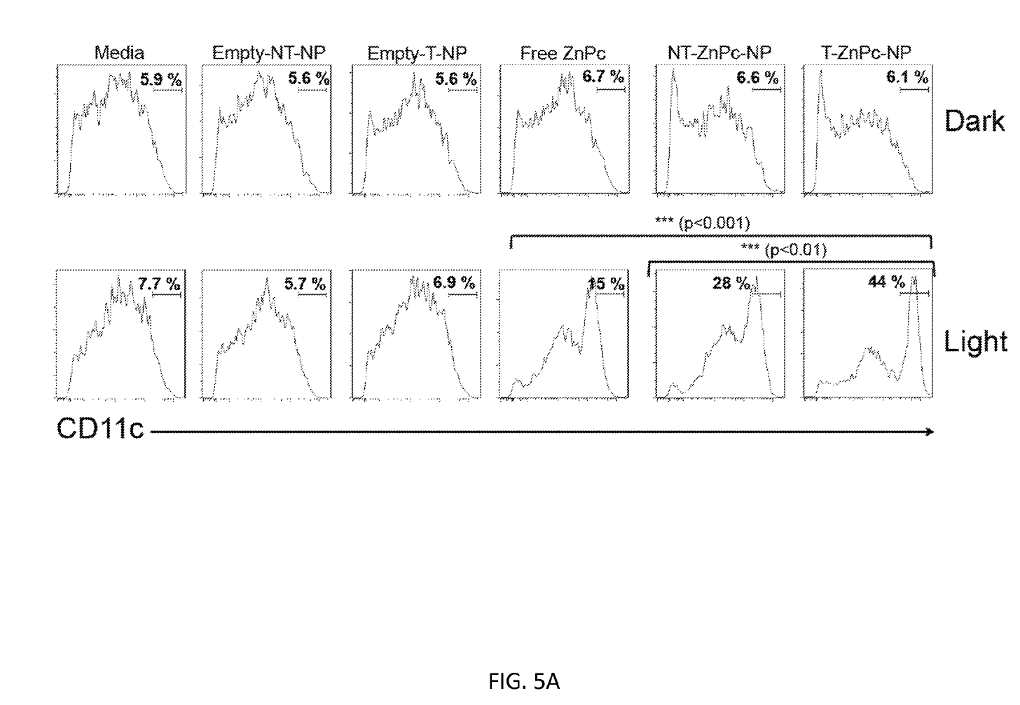

The ability of NPs to trigger immune activation was determined by incubating cells with targeted (T) or non-targeted (NT) ZnPc NPs, free ZnPc, empty targeted or non-targeted NPs, media, or lipopolysaccharide (LPS). Cells were exposed to 660 nm light for one minute. The effects of the NPs before and after exposure to light on IL-2, IL-4, IL-6, IL-10, TNF-alpha, and interferon-gamma (INF-gamma) were evaluated. The results are presented in FIGS. 4A (no light activation) and 4B (light activation). As shown, mitochondrial targeted ZnPc nanoparticles were generally more effective at activating certain aspects of an immune reaction than non-targeted ZnPc nonparticles, which were more effective than free ZnPc. Interestingly, the nanoparticles, particularly the mitochondrial targeted nanoparticles activated production of INF-gamma.

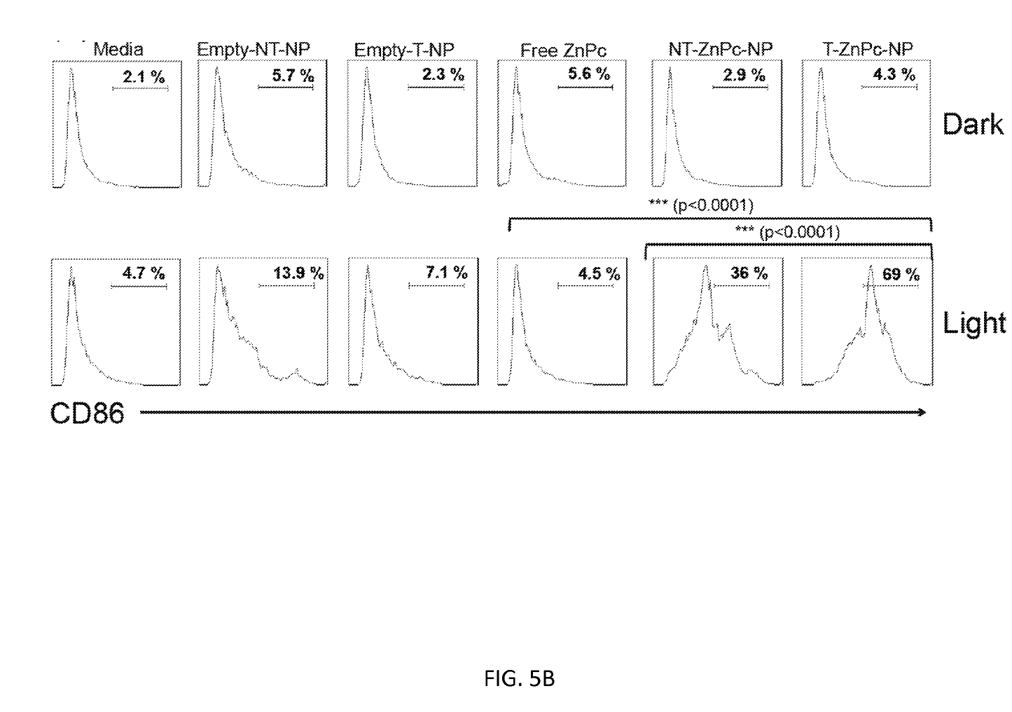

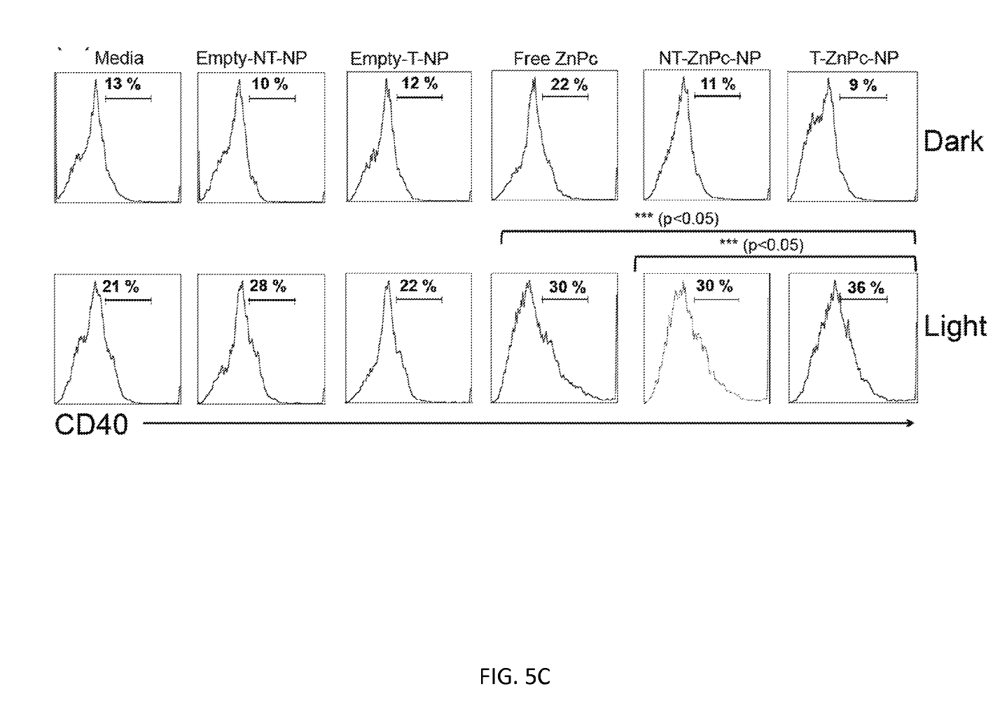

MCF-7 cells were treated with different conjugates (20 nM with respect to ZnPc for T-ZnPc-NP, NT-ZnPc-NP, and free ZnPc; Empty-NPs 0.5 mg/mL with respect to polymer) with or without subjected to light activation with a 660 nm LASER (20 mW) for 1 min, incubated for overnight at 37° C. under 5% CO2 in 10% FBS-DMEM media. The supernatants were harvested and incubated with BMDCs purified using anti-CD11c antibody for 24 h at 37° C. in 10% FBS-RPMI. Cells were harvested, washed with PBS, and stained with fluorescently labeled antibodies against CD11c (A), CD86 (B), and CD40 (C) and their surface expression was determined using flow cytometry. Data obtained were analyzed using flowjo software. QuickCalcs GraphPad student t test was used to calculate statistical significance. The results are presented in FIG. 5. As shown, MCF-7 cells stimulated with T-ZnPc-NP induces expression of CD11c, CD86, and CD40 markers on DCs.

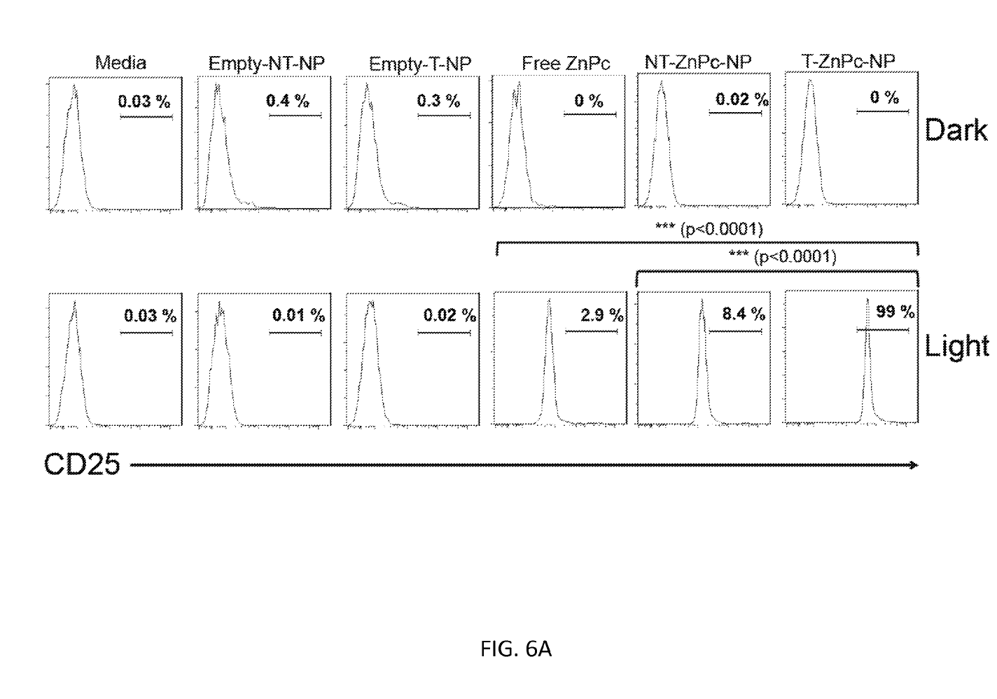

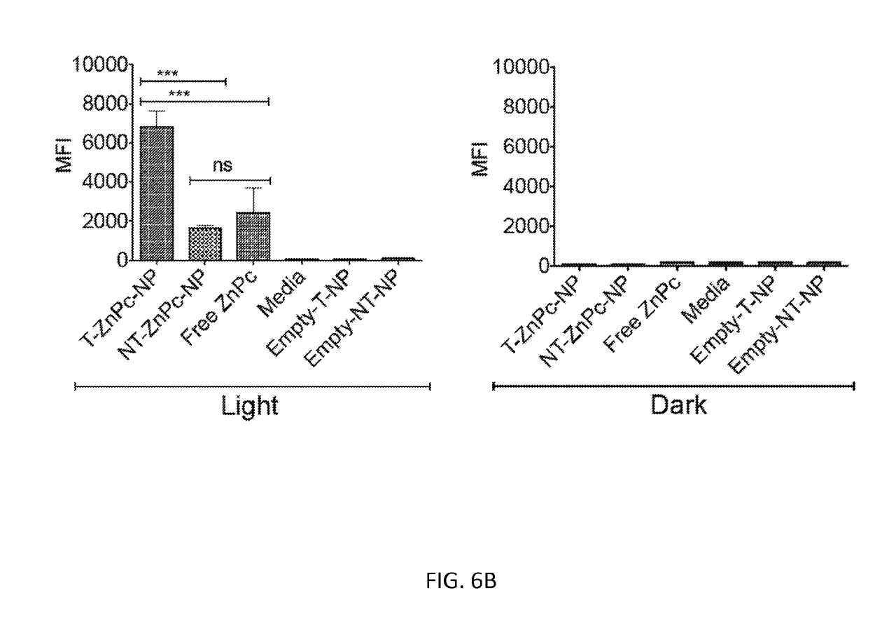

DCs were exposed to lysates of MCF-7 cells treated with different conjugates (20 nM with respect to ZnPc for T-ZnPc-NP, NT-ZnPc-NP, and free ZnPc; Empty-NPs 0.5 mg/mL with respect to polymer) with or without subjected to PDT with a 660 nm LASER (20 mW) for 1 min activates CD8+ T-cells. MCF-7 cells were incubated with different conjugates for 4 h with or without light activation followed by incubation for overnight at 37° C. in media containing 10% FBSDMEM. Cancer cell supernatants were harvested and incubated with BMDCs at 37° C. for overnight. BMDCs were washed with RPMI and further incubated with CD8+ T-cells enriched from spleens of C57BL/6 mice by negative selection method using macrobeads MACS column purification for 72 h at 37° C. Cells were harvested to measure T-cell activation by staining with antibodies against CD8 (surface marker) and CD25 (activation marker) using flow cytometry (A) and MFI of CD25 was calculated using flowjo software (B). QuickCalcs GraphPad student t test was used to calculate statistical significance. FIG. 6 shows the results of the CD8 T cell activation assays.

Thus, embodiments of GENERATION OF FUNCTIONAL DENDRITIC CELLS are disclosed. One skilled in the art will appreciate that the nanoparticles and methods described herein can be practiced with embodiments other than those disclosed. The disclosed embodiments are presented for purposes of illustration and not limitation.

1. A nanoparticle, comprising:
a mitochondrial targeting moiety; and
photosensitizer configured to produce a reactive oxygen species when illuminated with light having a particular wavelength.

2. A nanoparticle according to claim 1, wherein the photosensitizer is configured to produce a reactive oxygen species when exposed to light having a wavelength from about 600 nanometers to about 800 nanometers.

3. A nanoparticle according to claim 1, wherein the photosensitizer is a zinc pthalocyanin.

4. A nanoparticle according to claim 1, wherein the nanoparticle has a diameter of about 250 nanometers or less and has a zeta potential of about 0 mV or greater.

5-7. (canceled)

8. A nanoparticle according to claim 1, wherein the mitochondrial targeting moiety comprises a triphenyl phosophonium (TPP) moiety or a derivative thereof.

9-17. (canceled)

18. A nanoparticle according to claim 1, further comprising a cancer cell targeting moiety.

19-22. (canceled)

23. A method for treating a patient at risk or suffering from cancer, comprising administering a nanoparticle according to claim 1 to the patient.

24. A method for activating a bone marrow dendritic cell (BDMC), comprising:
contacting a cancer cell with a nanoparticle according to claim 1 and exposing the cancer cells to light within a wavelength that is configured to cause the photosensitizer to produce the reactive oxygen species; and
contacting a BDMC with the cancer cell or supernatant from the cancer cell that has been contacted with the nanoparticle and exposed to the light.

25. A method according to claim 24, wherein the cancer cells comprise breast cancer cells.

26-27. (canceled)

28. A method of producing IFN-gamma ex vivo from dendritic cells, comprising:
contacting dendritic cells with activated cancer cells or supernatant thereof to produce the IFN-gamma from the dendritic cells,
wherein activated cancer cells comprise cancer cells that have been contacted with a nanoparticle according to claim 1 and exposed to light of a wavelength that is configured to cause the photosensitizer to produce the reactive oxygen species.

29. A method according to claim 28, wherein the cancer cells comprise breast cancer cells.

30-31. (canceled)

32. A method for enhancing the immunogenicity of cancer cells, comprising:
contacting the cancer cells with a nanoparticle according to claim 1; and
exposing the cancer cells contacted with the nanoparticle to light of a wavelength that is configured to cause the photosensitizer to produce the reactive oxygen species.

33. A method according to claim 32, wherein the cancer cells comprise breast cancer cells.

34-35. (canceled)

36. A method for activating a bone marrow dendritic cell (BDMC), comprising:
contacting a cancer cell with a photosensitizer configured to generate a reactive oxygen species when exposed to light having a predetermined wavelength;
exposing the cancer cells that have been contacted with the photosensitizer to light of the predetermined wavelength; and
contacting a BDMC with the cancer cell or supernatant from the cancer cell that has been contacted with the photosensitizer and exposed to the light of the predetermined wavelength.

37. A method according to claim 36, wherein the cancer cells comprise breast cancer cells.

38-39. (canceled)

40. A method according to claim 36, wherein contacting the cancer cell with the photosensitizer comprises contacting the cancer cell with a nanoparticle comprising the photosensitizer.

41. A method according to claim 40, wherein the nanoparticle comprises a mitochondrial targeting moiety.

42. A method of producing IFN-gamma ex vivo from dendritic cells, comprising:
contacting dendritic cells with activated cancer cells or supernatant thereof to produce the IFN-gamma from the dendritic cells,
wherein activated cancer cells comprise cancer cells that have been contacted with a photosensitizer and exposed to light of a wavelength that is configured to cause the photosensitizer to produce a reactive oxygen species.

43. A method according to claim 42, wherein the cancer cells comprise breast cancer cells.

44-45. (canceled)

46. A method according to claim 41, wherein contacting the cancer cell with the photosensitizer comprises contacting the cancer cell with a nanoparticle comprising the photosensitizer.

47. A method according to claim 46, wherein the nanoparticle comprises a mitochondrial targeting moiety.

48. A method for enhancing the immunogenicity of cancer cells, comprising:
contacting the cancer cells with a photosensitizer; and
exposing the cancer cells contacted with the photosensitizer to light of a wavelength that is configured to cause the photosensitizer to produce a reactive oxygen species.

49. A method according to claim 48, wherein the cancer cells comprise breast cancer cells.

50-51. (canceled)

52. A method according to claim 48, wherein contacting the cancer cell with the photosensitizer comprises contacting the cancer cell with a nanoparticle comprising the photosensitizer.

53. A method according to claim 52, wherein the nanoparticle comprises a mitochondrial targeting moiety.
